(19) United States
(12) Patent Application Publication
(10) Pub. No.: US 2008/0233074 A1
(43) Pub. Date: Sep. 25, 2008

(54) Composition and Method For Dry Cow Udder Protection

(76) Inventor: Robert D. Kross, Bellmore, NY (US)

Correspondence Address: EDWIN D. SCHINDLER, FIVE HIRSCH AVENUE, P.O. BOX 966, CORAM, NY, 11727-0966, US

(21) Appl. No.: 10/599,259

(22) PCT Filed: Mar. 23, 2005
(86) PCT No.: PCT/US2005/009650

§ 371 Date: Sep. 23, 2006

Related U.S. Application Data
(60) Provisional application No. 60/555,562, filed on Mar. 24, 2004.

(51) Int. Cl. A61K 31/74; A61P 17/00

(52) U.S. Cl. 424/78.02

(57) ABSTRACT

A composition for dry cow udder protection includes a bimodal interpenetrating polymer system having both cationic and anionic functionalities and capable of forming a stable aqueous solution and ionic bonds between polar chains. The bimodal inter-penetrating polymer system, preferably, includes two acrylate copolymers, Polyacrylate-18 and Polyacrylate-19. The bimodal interpenetrating polymer system is approximately 20% to approximately 40%, by weight, of the aqueous solution, and preferably has a thixotropic viscosity of from approximately 500 cps to approximately 5,000 cps, as measured with a Brookfield Viscometer at 20 rpm with a #3 spindle. The composition, as part of an aqueous solution, is applied to the region of a cow teat to be protected and allowed to dry, resulting in a water-insoluble protecting film.

### CROSS-REFERENCE TO RELATED U.S. APPLICATION

### CROSS-REFERENCE TO U.S. PROVISIONAL PATENT APPLICATION

The inventor claims priority on the basis of U.S. Provisional patent Application No. 60/1555,562, filed Mar. 24, 2004, the entire disclosure of which shall be deemed to be incorporated by reference herein.

### BACKGROUND OF THE INVENTION

1. Technical Field of the Invention

The present invention relates, generally, to a composition and related method for dry cow udder protection.

More particularly, the present invention relates to a composition and related method for dry cow udder protection which utilizes various bimodal interpenetrating polymer networks which comprise both cationic and anionic functionalities, which form stable aqueous solutions.

During the drying process, the bimodal interpenetrating polymer networks rapidly interact with one another by forming ionic bonds between polar chains and become ionically cross-linked. Such systems, upon drying, whether on mammalian tissue or inanimate substrates, form water-insoluble films that adhere to the surface upon which they have dried and have been found to be quite useful for dry cow udder protection.

2. Description of the Prior Art

The dry, or non-lactating, period of a cow is the approximately four- to ten-week period immediately preceding the delivery of a calf. Although a cow's normal lactation period is about 300 days per year, it has been estimated that forty- to fifty-percent of teat infections occur during the cow's dry period. This high rate of infection occurs because the cow has a diminished immune response during the dry period, as well as, because the teat is distended during the dry period for facilitating the penetration of the mammary gland by mastitis-causing organisms. Without a daily flushing by the milking process, infecting microorganisms are more likely to implant and proliferate. As a result, so-called “dry-cow therapy” has become an essential component of a mastitis control program.

Dry-cow therapy often involves the treatment of the udder with medication, which can beneficially remain within the udder tissue for extended periods without the medication having to be discontinued several days prior to milking time, so as to avoid residue from the medication in the milk during the cow's lactation period. Such extended treatment would therefore minimize the rate of udder infections. If the cow's health can be restored during its dry period, treatment with antibiotics during its lactating period, which reduces the potential for residues of therapeutic agents during its lactation period, may not be necessary.

During the active lactation period, mastitis is most easily controlled by using germicidal pre- and post-milking teat dip compositions. Such germicidal dips kill bacteria that are introduced onto the surface of the animal from many sources, including milking machines, the milker's hands, its bedding and a host of other environmental sources. Such other environmental sources include bacteria that can impinge upon, and remain on, the cow's teats during the entire period between milkings (which might be approximately 12-14 hours, at times.)

The post-milking teat dips often include a film-forming agent, as well as a germicide. The film, or barrier, is intended to deposit an extra protective layer on the teat and is designed to have a sufficient retentive capacity to last through the inter-milking period, but is nevertheless readily removable when the cow's teats are cleaned prior to subsequent milkings. This form of protection represents an often difficult balancing act between making the barrier film sufficiently resistant to environmental moisture, such as to mud and rain, and yet having sufficient water solubility that the protective layer can be readily removed during the pre-milking, water-rinse teat preparation.

One such soluble barrier is the poly(acrylamido methane-sulfonic acid) polymer found in the currently marketed “UdderGold” (Trademark) series of teat dips. Another soluble barrier is polyvinyl alcohol, which is used in a number of teat dips. The former material enhances the viscosity of the teat dip; the latter does not.

Generally, and depending upon environmental conditions, the protective films that form upon drying of the dip can wear off in about 3 to 4 hours. Dissolution and/or deterioration of the film is usually greatest when conditions are wet and the barriers are, as a result, more-readily removable. Under such conditions, the environmentally-associated bacteria are more likely to proliferate and have a greater potential for infection.

During the dry-period, however, it is possible to make use of a less water-soluble film material, so that the deposited film can remain in place for days or weeks, even under adverse climactic conditions. Such film would, most importantly, form a plug at the teat end opening and thereby be a physical obstacle to the penetration of infectious bacteria. This film, as for the pre- and post-milking dips, would also be formulated to contain an antimicrobial material or combinations thereof. The germicidal action of the antimicrobials(s) would not necessarily be as rapid, or as powerful, as those that are used for the shorter-contact pre-and post-milking dips, since they would be in place for greater time periods. There are many antimicrobials that can fit into this category, including most of the single-phase systems in current use for lactating-associated dips, in addition to others, which may be slower-acting, but nevertheless appropriate for the dry-dip application.

Erhard et al., U.S. Pat. No. 6,440,442, issued Aug. 27, 2002, teaches a dry period teat dip comprising a dual polymer system with a first component being a solvent-soluble, pre-formed, thermoplastic polyurethane and a second component, a polymer, being a hydrophilic poly(N-vinyl lactam); the blend, upon evaporation of solvent, being capable of forming a water-resistant film upon topical application to mammalian skin without appreciable loss of the poly-(lactam) through moisture in the environment. The prior art composition also contains at least one antimicrobial agent, seemingly capable of being removed via peeling.

In earlier years, a latex-based dry dip was available, although antimicrobials were generally not compatible with these materials, and such barriers actually fomented the growth of bacteria between the barrier and the skin. Other recent coatings considered for teat dip application include polyvinylpyrollidone and other vinyl polymers, protein hydrolyzate, and natural and synthetic gums.

A further product in current use is a paste, Orbeseal (Trademark), which purportedly “provides a malleable barrier in the teat canal” for preventing bacteria from entering the teat canal during the dry period. This product is infused, by syringe, into each quarter and is subsequently removed by stripping before calving, or ingested by the calf, or eliminated, during milking. It can, in the latter situation, cause blockage in the milking machine. This treatment is cumbersome to apply and eliminate, and currently costs about $2 to treat each cow.

Other dry cow therapies that are standard in the industry include teat dip compositions that contain strong solvents, some of which (e.g., tetrahydrofuran) are cytotoxic and cause irritation to skin, eyes and the respiratory tract. The irritation to skin includes symptoms such as redness, itching, rash, cracking and pain. Tetrahydrofuran is harmful if swallowed, or inhaled, is an extremely flammable liquid, and repeated doses may cause kidney or liver damage. Tetra-hydrofuran may also affect the lungs and central nervous system.

The product disclosed by Erhard et al., U.S. Pat. No. 6,440,442, is a complex mixture of materials, relying on the physical entrapment of a soluble protective polymer within the structure of another polymer. This prior art film is regarded as “water-resistant,” rather than water-insoluble, so that it can slowly dissolve in the presence of excessive environmental moisture and thereby provide reduced protection. As a result of its complexity, the price for the commercial product, T-Hexx (Trademark), is currently over $70 per quart.

The present invention is the result of a search for a dry dip composition that forms an insoluble film on the teat, following application of a solubilized precursor polymer composition, which composition is economically more beneficially priced as compared to current dry dips.

### SUMMARY OF THE INVENTION

It is, therefore, an object of the present invention to provide a composition and method for dry cow udder protection that will rapidly dry following application to form a water-insoluble film for providing udder protection.

It is a further object of the present invention to provide a composition and method for dry cow udder protection that provide a film capable of withstanding the presence of excessive environmental moisture and other extreme conditions that would otherwise undermine prior art compositions and methods used for dry cow udder protection.

It is, yet, an additional object of the present invention to provide a composition and method for dry cow udder protection that is cost-effective, as compared to currently known compositions and methods for dry cow udder protection.

The foregoing and related objects are accomplished by the present invention of a composition and related method for dry cow udder protection, which include various polar acrylate solutions which are capable of forming insoluble films upon drying of their aqueous components. More particularly, these polar acrylate solutions are comprised of bimodal interpenetrating polymer networks, which include both cationic and anionic functionalities that are capable of forming stable aqueous solutions. Upon the drying of such solutions, the bimodal interpenetrating polymer networks rapidly interact with one another by forming ionic bonds between their polar chains, thereby becoming ionically cross-linked to provide a water-insoluble film for udder protection.

The polymer compositions of the present invention are produced by a polymerization process by which the polymers are synthesized in the presence of one another. A particularly preferred polymer composition is the system categorized by the International Nomenclature of Cosmetics Ingredients (“INCI”) name as “Polyacrylate-18” and “Polyacrylate-19,” and are comprised of two acrylate copolymers. Such polymer systems, upon drying, whether on mammalian tissue or inanimate substrates, form water-insoluble films that adhere to the surface upon which they have dried.

Generally, the solids contents of these particularly preferred aqueous polymer solutions range from about 20% to about 40% by weight. The films of the present invention are inherently water insoluble under environmental conditions characteristic of those which confront mammalian species, particularly cows and goats. The water insoluble films can remain on the animal for many days, particularly in the teat opening in which the solution would flow, accumulate and evaporate in larger quantities than on the sides of the teat. The films would remain substantially intact, despite exposure of the animal to environmental moisture such as rain, dew, ponds and mud. Additionally, these films are moisture-vapor permeable, and permit transpiration of gases and other volatile physiological compounds, which are necessary for proper functioning of mammalian skin. If necessary, the compositions may be physically removed and, over time, if the film has been removed, may again be applied to restore the protective coating.

Other objects and features of the present invention will become apparent when considered in view of the following detailed description of the invention, which provides certain preferred embodiments and examples of the present invention. It should, however, be noted that the accompanying detailed description is intended to discuss and explain only certain embodiments of the claimed invention and is not intended as a means for defining the limits and scope of the invention.

### DETAILED DESCRIPTION OF THE PREFERRED EMBODIMENTS

The viscosity of the bimodal interpenetrating polymer compounds of the present invention may be partially controlled by an upward pH adjustment so as to increase the relative amount of acrylate anion versus acrylic acid functionality. Those skilled in the art of polymer compounding and cosmetic formulation would be familiar with appropriate agents to effect such pH modification, wherein such agents would include, for example, ammonium salts and compounds of the ethanolamine family, as well as alkali and alkaline earth hydroxide compounds, such as, e.g., sodium and calcium hydroxide.

The compositions may be suitably thickened, as well, by the use of appropriate thickening agents that are known to the skilled artisan of compounding. Such thickening agents would include, for example, members of the cellulosic family, such as, e.g., sodium carboxymethyl cellulose, members of the Carbopol family, such as, for example, Carbopol 960, inorganic thickeners, such as, e.g., the members of the hydrated silica family and a range of natural and synthetic thickening agents, such as, e.g., the xanthan gums, polyacrylamides and members of the family thereof, such as, for example, the sodium salt of polyacrylamido methanesulfonic acid.

A number of dermatologically-compatible solvents may be incorporated into these acrylate solutions, at varying and appropriate degrees, for enhancing the rapidity of evaporation of the film on the teat skin. These solvents should be non-cytotoxic and nonirritating to mammalian skin. Examples of such solvents include ethanol, isopropanol, ethyl lactate, diacetone alcohol, N-methyl pyrrolidone and mono- and di-ethylene glycol ethers. Ethanol and isopropanol are the preferred solvents. Examples of antimicrobial agents used in the present composition include iodine, chlorhexidine, sodium dodecylbenzene sulfonate, nitrous acid, bronopol and triclosan.

In a preferred embodiment, the composition demonstrates thixotropic viscosity characteristics, and ranges in viscosity from about 500 to about 5000 cps, when measured with a Brookfield Viscometer at 20 rpm with a #3 spindle. This range in viscosity allows an adequate amount of the composition to deposit and remain on the mammalian teat, with low drip loss. After the mammalian teats have been coated with the composition of the present invention, the resulting coating is permitted to dry to an adherent solid film on the teats. Typically, some of the still-liquid coating material flows down to the teat end where a plug-like deposit is formed for effectively sealing off the teat canal.

Variations in formulations for a dry-cow teat dip from an aqueous bimodal polymer dispersion of the present invention can be best illustrated through reference to the following Examples. These Examples and data provide a basis for understanding the metes and bounds of the invention and are not to be taken as a limitation upon the overall scope of the present invention.

### EXAMPLE 1

Example 1 is an example of a dry-cow teat dip prepared from an aqueous bimodal polymer dispersion, Syntran EX-100 from the Interpolymer Corp., which contained about 25% of polymer components. This dispersion represented the major component of the dip, as follows:

| Components | |
|---|---|
| (in order of addition) | Percentage |
| Carbitol | 1.00 |
| Polyethylene Glycol-600 | 3.00 |
| Xanthan Gum | 0.51 |
| Veegum PRO | 2.00 |
| Water | 5.00 |
| Dodecylbenzene Sulfonate, Sodium | 0.20 |
| EX-100 Polymer Dispersion | 88.26 |
| Chroma-Lite Red | 0.03 |

The dry dip formulation contained xanthan gum and Veegum silica thickeners to create a thixotropic composition which resists significant drippage loss following immersion. The composition had a viscosity of 1,055 cps, when measured with a Brookfield Model RVT viscometer, and a #3 spindle at 10 rpm. The use of the dodecylbenzene sulfonate component contributed antimicrobial activity to the composition and the Chroma-lite provided an enhanced visibility of the dried dip film. The polyethylene glycol imparts a flexibility to the dried film, which is otherwise too brittle. When the teats of a cows udder are briefly immersed in this mixture, a strongly adherent and coherent film forms within several minutes and, once formed, is very difficult to remove manually. Protection is afforded to the dipped teat for several weeks, with respect to the infusion of environmental pathogens.

### EXAMPLE 2

Example 2 is an example of a faster-drying film than that produced by the dip of Example 1, which allows the cow to return to the field more rapidly without the concern of environmental components adhering to the still-damp dip film. The composition of the dip is as follows:

| Components | |
|---|---|
| (in order of addition) | Percentage |
| Polyethylene Glycol-600 | 3.00 |
| Dodecylbenzene Sulfonate, Sodium | 0.20 |
| EX-100 Polymer Dispersion | 96.75 |
| Chroma-Lite Red | 0.05 |

A thinner film is formed on the teat, upon drying, with a less intense color. The dried film adheres very tenaciously to the teat skin, is flexible and can deform without losing integrity as the teat skin is flexed.

### EXAMPLE 3

Example 3 is an example of a dry-cow teat dip prepared from Interpolymer's aqueous bimodal polymer dispersion, Syntran EX-104, which contained about 35% of polymer components. No additional water was used in the formulation. The xanthan gum thickener and dodecylbenzene sulfonate were pre-dispersed in the PEG-600, after which the EX-104 was added with stirring. While stirring, the yellow colorant was added, and the mixture was stirred until its full viscosity developed.

| Components | |
|---|---|
| (in order of addition) | Percentage |
| Polyethylene Glycol-600 | 3.00 |
| Xanthan Gum | 0.50 |
| Dodecylbenzene Sulfonate, Sodium | 0.20 |
| EX-104 Polymer Dispersion | 96.00 |
| FD&C Yellow #5 | 0.30 |

The viscosity of this dry dip formulation was 600 cps, as measured with a Brookfield viscometer using a #3 spindle at 20 rpm. The dry, antimicrobial film is adhesive to the teat skin for many days, with no loss of integrity upon normal flexure.

### EXAMPLE 4

Example 4 is an example of a faster drying dry-cow teat dip prepared from Interpolymer's aqueous bimodal polymer dispersion, Syntran EX-107. The latter was diluted with anhydrous isopropyl alcohol, at a 70:30 ratio, and to that mixture was added 0.10% benzoic acid (as an antimicrobial), 2.0% polyethylene glycol (to reduce film brittleness), and 0.1% FD&C Blue #1 (for film visualization). The resulting composition is discernibly viscous and has a low tendency to drip after application. The film resulting from drying of this mixture on an animal's teats is relatively tack-free in 5-10 minutes after immersion therein. The film is additionally flexible enough to conform to normal skin stresses for the days and weeks that the film remains on the teat.

### EXAMPLE 5

Example 5 is an example of a more rapid drying dry-cow teat dip prepared from Interpolymer's aqueous bimodal polymer dispersion, Syntran EX-107-20, which is a more viscous version of the material used in Example 4. This material was diluted with anhydrous isopropyl alcohol, at a 80:20 ratio, and to that mixture was added 0.10% benzoic acid (as an anti-microbial), and a mixture of 0.1% FD&C Blue #1 and Yellow #5, to create a green deposit upon drying. The resulting composition is more viscous than the mixture of Example 4, with an even lower drip tendency. The dried film is tack-free within 5 minutes on the teat side, with a drop on the teat bottom that dries within about 10 minutes. The film is flexible enough, without plasticizer, to conform to normal skin for an extended time after application.

While only several embodiments of the present invention have been shown and described, it will be obvious to those skilled in the art that many modifications may be made to the present invention without departing from the spirit and scope thereof.

What is claimed is:

1. A composition for dry cow udder protection, comprising:
a bimodal interpenetrating polymer system having both cationic and anionic functionalities and capable of forming a stable aqueous solution and ionic bonds between polar chains.

2. The composition for dry cow udder protection according to claim 1, wherein said bimodal interpenetrating polymer system is comprised of Polyacrylate-18 and Polyacrylate-19.

3. The composition for dry cow udder protection according to claim 1, wherein said bimodal interpenetrating polymer system is approximately 20% to approximately 40%, by weight, of an aqueous solution.

4. The composition for dry cow udder protection according to claim 1, wherein said bimodal interpenetrating polymer system is in an aqueous solution having a thixotropic viscosity of from approximately 500 cps to approximately 5,000 cps, as measured with a Brookfield Viscometer at 20 rpm with a #3 spindle.

5. The composition for dry cow udder protection according to claim 4, wherein said aqueous solution includes a dermatologically-compatible solvent for enhancing rapidity of evaporation of said aqueous solution on a cow teat.

6. The composition for dry cow udder protection according to claim 5, wherein said dermatologically-compatible solvent is non-cytotoxic and non-irritating to mammalian skin.

7. The composition for dry cow udder protection according to claim 5, wherein said dermatologically-compatible solvent is a member selected from the group consisting of ethanol, isopropanol, ethyl lactate, diacetone alcohol, N-methyl pyrrolidone, a monoethylene glycol ether, a diethylene glycol ether and a combination thereof.

8. The composition for dry cow udder protection according to claim 4, wherein said aqueous solution includes an antimicrobial agent.

9. The composition for dry cow udder protection according to claim 8, wherein said antimicrobial agent is a member selected from the group consisting of iodine, chlorhexidine, sodium dodecylbenzene sulfonate, nitrous acid, bronopol, triclosan and a combination thereof.

10. A method for dry cow udder protection, comprising the step(s) of:
applying an aqueous solution containing a bimodal interpenetrating polymer system to a cow teat.

11. The method for dry cow udder protection according to claim 10, wherein said bimodal interpenetrating polymer system includes Polyacrylate-18 and Polyacrylate-19.

12. The method for dry cow udder protection according to claim 10, wherein said bimodal interpenetrating polymer system comprises approximately 20% to 40%, by weight of said aqueous solution.

13. The method for dry cow udder protection according to claim 10, further comprising the step of:
adjusting viscosity of said aqueous solution for allowing an adequate amount of said bimodal interpenetrating polymer system to deposit, dry to a film, and remain on the cow teat.

14. The method for dry cow udder protection according to claim 13, wherein said viscosity of said aqueous solution has a thixotropic viscosity of from approximately 500 cps to approximately 5,000 cps, as measured with a Brookfield Viscometer at 20 rpm with a #3 spindle.

15. The method for dry cow udder protection according to claim 13, wherein said step of adjusting viscosity of said aqueous solution is carried out by adjusting pH of said aqueous solution.

16. The method for dry cow udder protection according to claim 15, wherein pH is adjusted with a member selected from the group consisting of an ammonium salt, an ethanolamine family composition, an alkali earth hydroxide composition, an alkaline earth hydroxide composition and a combination thereof.

17. The method for dry cow udder protection according to claim 15, wherein pH is adjusted with a member selected from the group consisting of sodium hydroxide, calcium hydroxide and a combination thereof.

18. The method for dry cow udder protection according to claim 13, wherein said step of adjusting viscosity of said aqueous solution is carried out by adding a thickening agent to said aqueous solution.

19. The method for dry cow udder protection according to claim 18, wherein said thickening agent is selected from the group consisting of a cellulosic composition, a Carbopol composition, a hydrated silica composition and a combination thereof.

20. The method for dry cow udder protection according to claim 18, wherein said thickening agent is a natural or synthetic thickening agent selected from the group consisting of a xanthan gum, a polyacrylamide, a salt of a polyacrylamide and a combination thereof.
